(19) United States
(12) Patent Application Publication
(10) Pub. No.: US 2021/0015951 A1
(43) Pub. Date: Jan. 21, 2021

(54) SYSTEM FOR UV STERILIZATION WITH INTEGRATED IMAGING AND REPORTING SYSTEM

(71) Applicant: Lumagenics, LLC, Nashville, NC (US)

(72) Inventors: Amy V. Harris, Apex, NC (US); Charles G. Gillooly, Wake Forest, NC (US)

(73) Assignee: Lumagenics, LLC, Nashville, NC (US)

(21) Appl. No.: 16/511,797

(22) Filed: Jul. 15, 2019

(51) Int. Cl. A61L 2/10; H04N 13/204; G06T 11/20

(52) U.S. Cl. CPC A61L 2/10; H04N 13/204; G06T 2200/24; A61L 2202/11; A61L 2202/14; G06T 11/20

(57) ABSTRACT

System for sterilization which includes imaging device in register with a UV light to allow a user to visually see the surface sterilized and surfaces that are missed by displaying on a graphic user interface.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a system for UV sterilization of surfaces. In particular, it relates to a UV sterilization system that tracks where UV light has been used and if sufficient dose has been administered.

### Description of Related Art

Microbiological sterilization has been pivotal in the production of many products with extended storage times. Various technologies have been developed to achieve this sterilization, including UV-irradiation both high and low energy, gamma-ray irradiation (or gamma irradiation), chemical sterilization, heat sterilization, autoclaving, and ultrafiltration. Because these technologies can destroy both microorganisms and various substrates or surface materials, they have experienced limited adoption to date. In light of this fact, a particular technology may not always be acceptable for sterilizing a given product. Recently, an increase in the number and variety of products has created a need for adequate sterilization without the damaging side-effects to the desirable and heat sensitive components of the product. Chemical sterilization, heat sterilization, and autoclaving all damage or alter biological molecules and plastics. The inactivation of biological molecules effectively kills the microbe that utilized these molecules for life processes.

Ultrafiltration, a recent technology relative to the others mentioned here, requires the use of filters with a very minute pore-size (at least <0.45 microns). These filters are an inherently slow means of sterilization, and may not be suitable for solutions of high viscosity, solutions, or surfaces that contain desirable particles that are larger than the pore diameter and, consequently, too large to pass through the filter. Gamma-irradiation is a technology not commonly used for sterilization. One major reason for its lack of widespread use is that it utilizes a radiation source, such as radioactive cobalt, that is very radioactive, and thus, very dangerous. This technology requires extensive shielding and control systems to prevent accidental exposure to operators and others. These protective requirements are economically expensive, often prohibitively so. Therefore, gamma-irradiation is often not an economically acceptable technology or a safe technology for sterilization of products. This photochemical mechanism of sterilization may also degrade the desired product, rendering it inactive or damaged, and thus defeating the purpose of the sterilization.

UV-irradiation has been used extensively for sterilization. Generally, devices that use UV light to sterilize products are composed of one or more of a power supply (ballast), a UV light source, a light-focusing and/or light-conducting device, a light filter, and a control system to assure proper operation. The ballast is designed to supply power to the lamp in a reliable fashion in order to ensure continuous optimal function of the lamp. A variety of UV light sources exist and are known in the prior art, including pulsed, gas-filled flash lamps, spark-gap discharged apparatus, LED, or low-pressure and high pressure mercury vapor lamps. Traditionally, low-pressure mercury vapor lamps have been used for sterilization because these lamps are relatively inexpensive to operate and emit relatively higher amounts of UV irradiation than other sources. Other types of vapor lamps are also used, including mercury-xenon (HgXe) lamps, simple fluorescent, or LED light are embodiments.

By way of background, light is conventionally divided into infrared light (780 nm to 2600 nm), visible light (380 nm to 780 nm), near UV light (300 nm to 380 nm), and far UV light (170 nm to 300 nm). Most UV lamp sources emit light at discrete wavelengths and include filters to filter out or block wavelengths other than the specific UV wavelength, especially 254 nm. In the UV region, the most notable UV emission occurs at 254 nm. It is known that mercury vapor lamps emit radiation at 254 nm. This wavelength can damage the genome of cells and viruses, thus inhibiting their replication, thereby sterilizing the cells and viruses. Therefore, generally in the prior art, a single wavelength detector tuned to 254 nm has been used to determine the amount of UV radiation reaching the target. In order to optimize the UV light output efficiency of the lamp source, at least one filter was interposed in the light path in order to block non-UV light from reaching the target, allowing only UV and proximate-UV light to reach to target. Therefore, the industry has evolved over time with the solidly established paradigm that 254 nm is the sole and exclusive wavelength of importance for UV sterilization. As such, the prior art teaches away from the inclusion of non-UV wavelength light for microbial sterilization apparatus. Furthermore, this paradigm not only teaches that polychromatic or broad spectrum light as irrelevant or unimportant, but disadvantageous.

More recently, UV light systems for sterilization using a wand or other hand-held device are one way used to deliver UV light to a surface substrate. In use, one shines the UV light with or without a wand on a portion of the surface to be sterilized and moves the wand device around until the entire surface has been treated. Even more recent is the development of high intensity UV lights wherein the UV light is delivered to the substrate.

A serious problem with this technology is making sure the entire surface is actually treated without missing portions and allow for sufficient time and energy to sterilize the entire surface. Depending on the user's skill, this could easily lead to a missed portion of the surface, leading to more serious infections. Currently, there is no solution for this problem. In addition, there is no record of if the treatment was done nor the time to accomplish the process. Even further, with current UV systems, there is a complete lack of auditing capabilities, reporting, user feedback, and the like.

### BRIEF SUMMARY OF THE INVENTION

The present invention relates to combining a 2D or 3D modeling scanner, i.e. an imager, in lock register with a sterilizing UV source that is used to paint and sterilize a surface with sterilizing UV light source such that a graphic representation of where the surface has been painted is produced in real time. Time, energy levels, distance, and the like are provided to create a record and make sure sufficient light is used. Any missed areas of the surface can then be sterilized as well. In one embodiment, the system can also track exposure time, distance or the light to make sure not only is the entire substrate surface covered, it has been done for sufficient time, distance, or the like, to complete the sterilization of the entire surface.

Accordingly, in one embodiment, there is a real time system for sterilization of a substrate surface comprising:
a) a UV light source with a UV emitter designed to paint the substrate surface with sterilizing UV light of a given area on the substrate surface when the UV light is shined on the substrate surface;
b) an imaging device having a measuring laser, light, camera, or the like such that the imaging device creates data therefrom;
c) a computer graphics program for converting the scanning device data into an image of the surface of the substrate that has been scanned; and
d) a user interface for depicting the image of the surface of the substrate that has been scanned and creates data about the sterilization.

In another embodiment, there is a method of sterilizing a substrate surface with UV light comprising:
a) selecting a UV light source with a UV emitter designed to paint the substrate surface with sterilizing UV light of a given area on the substrate surface;
b) selecting an imaging device which when sterilizing the substrate surface with the UV light source is being done, the scanner scans where the substrate has received UV light and creates data therefrom about the method;
c) painting the substrate surface with the UV light source while simultaneously scanning the substrate surface with the imaging device to create data therefrom about the method;
d) providing computer graphics software that converts the imaging device data into an image; and
e) providing the image to a user graphic interface which allows the user to view at least one of what parts of the substrate surface has been scanned, what has not been scanned, and data generated by the imagery device.

### BRIEF DESCRIPTION OF THE DRAWINGS

FIG. 1 is a graphic depiction of the system of the invention.

FIG. 2 is a flow chart of the method of the present invention.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “system for sterilization of a substrate surface” refers to a system that has a light source producing a UV sterilizing light either wide spectrum or narrow spectrum (e.g., UVA, UVB, and UVC) of light capable of killing a microorganism, such as a bacteria or virus that is on a solid or liquid substrate. In one embodiment, it produces a wide UV spectrum (i.e., more than just an isolated wavelength) even though it can produce other spectrums of light and, in another embodiment, the light produces a high energy UV output with or without heat. Solid substrates are anticipated for sterilization using this system. Essentially, inanimate objects that are not moving are contemplated as substrate surfaces. In one embodiment, the substrate is an implantable device, patient contact item, patient care items, shared equipment, common surfaces, and the like.

As used herein, the term “sterilization” refers to effectively sterilizing the entire substrate surface i.e., very little residual contamination remaining, in one embodiment less than 1%. Other embodiments include orthopedic replacements, bed rails, bedside tables, phones, call buttons, chairs, door handles, computer keyboards, IV poles, light switches, sinks, toilets, handrails, and the like.

As used herein, the term “substrate surface” refers to a solid surface inanimate objects like a table, chair, wall, floor, and the like which is to be hand sterilized by utilizing a hand-held UV sterilizing source of light.

As used herein, the term “paint” refers to how hand-held UV sterilization devices are used. A user has a hand-held UV emitter and shines the beam for the emitter back and forth across the substrate surface essentially painting or moving back and forth with light until the entire surface has been painted. Because sterilization is a function of time, distance, light energy, and other factors, in one embodiment, the system also measures parameters that would measure if each part of the substrate surface has received enough UV light to be efficiently sterilized.

As used herein, the term “UV light source” refers to a bulb of any kind which produces a sterilizing UV light. Regular bulbs, LED, or other low energy bulbs are within the scope of the invention, but high intensity discharge (HID) bulbs are also embodiments of the invention. So, for example, a high energy mercury xenon (HgXe) bulb can be utilized. These types of bulbs are high UV output bulbs. In general, the light output of high energy bulbs of the invention are from about 0.1 J/cm² to about 50.0 J/cm².

As used herein, the term “UV emitter” refers to a device that collects light from a UV light source and focuses the light into a stream. The light then terminates in a hand-held portion or devices where the light is emitted and then directed to the substrate surface via the hand-held portion of the UV emitter. The focusing creates a higher intensity and focused light output. The focus can be electric powered or have a manual way to focus the light. In one embodiment, it includes a system for removing the heat prior to the light emitting from the UV emitter.

As used herein, the term “high energy light output” refers to light output of about at least 80 lumens per watt output in a high pressure lamp. In order to achieve this light output, one cannot use low or medium pressure lamps that produce UV light, as they do not produce enough light output. An arc discharge lamp does not produce the level of light output intensity needed. In order to achieve the high intensity output needed, one can add to the arc discharge lamp's light output an elliptical reflector which collimates the polychromatic light into still greater intensity (intensity being understood as energy per area) of about 100 lumens per watt (i.e., producing the high intensity light output needed). In other embodiments, the invention works with any UV light including LED's, medium pressure lamps, low pressure lamps, and the like.

As used herein, the term “power supply” refers to an AC or DC source that powers the light supply and, where needed, the optical device or any other part of the device.

As used herein, the term “polychromatic” refers to light comprising multiple wavelengths of light. In other embodiments, one or more of UVA, UVB, and UVC are contemplated.

As used herein, the term “exposure period” refers to the time period that light produced by the system is shown on a substrate. In one embodiment, it is from about 0.01 seconds to about 5 seconds for a high energy beam. In one embodiment, a shutter is utilized to open, close, and modulate the passage of light from the light source to the microorganism. This time is for each area exposed to the light. The size of the substrate will determine the entire time to sterilize the surface. Since energy falling on a substrate is a function of distance, the distance of the emitter from the substrate can be measured.

As used herein, the term “light guide” refers to a device for taking light emitting from the bulb and helping to deliver it to the UV emitter. While the guide is not necessary to use the invention, it is an embodiment that helps focus or make it easier to deliver the focused light to a desired location/substrate, and the like. The guide is generally a tube having a first and a second end of the tube, wherein the first end is used to collect light outputting from the device when positioned proximate to the device, such that the light channels through the tube and is delivered to the second end and out thereof, to deliver light where desired, e.g., a substrate. The light guide could include a collimator. The light guide can be at least one of: flexible, UV transmissive, have or be a liquid, have an aqueous salt solution, have a metallic salt solution, wherein, in one embodiment, the metallic salt is Na, K, Mg or combinations thereof, a non-aqueous solution, or a gas.

As used herein, the term “UV light” refers to the UV output of the selected UV lamp.

As used herein, the term “distance” refers to the distance between the UV emitter and the substrate surface.

The present invention generally relates to microbial sterilization (or DNA disruption or DNA inactivation), and more particularly to microbial sterilization using a UV light source on a substrate surface. One of the objectives of the present invention is to improve on the prior art by more effectively sterilizing a substrate without missing or under or over radiating any portion of the substrate surface. Excessive denaturing of any surface can be minimized by selecting a high energy, low heat UV light source. A further object of the present invention is the use of a shutter mechanism for the modulation of the exposure. A further embodiment of the present invention is the use of a dichroic reflector for removal of thermal energy and the focusing (concentrating) of light. A further object of the present invention is the use of an electronic circuit board for modulating lamp power, thermals, and shutter timer.

Thus, it is one embodiment of the present invention to provide a system that will efficiently sterilize a substrate surface with a hand-held UV emitter.

As used herein, the term “scanning device or “imaging device” refers to a 2D or 3D process of capturing digital information about the shape (surface) of an object with equipment that uses a laser, light, camera, or other digital means and to measure the distance between the scanner and the object, areas sterilized, and the like. This is also known as Imaging, Laser Scanning, Laser Digitizing, and Digital Shape Sampling & Processing (DSSP), as well as others.

As used herein, the term “lock register” refers to the camera, laser or light being focused on the identical portion of the substrate surfaces as is being painted by the UV light source. In this way, the graphic image created is also congruent with the surface area treated by the UV light source.

As used herein, the term “data” refers to information generated to be delivered to a user for both a record of activity as well as the efficiency of the process, such as UV exposure period, UV light energy, distance to substrate, auditing capabilities, management reporting (dose, time, distance, location, user, date, real time), and the like.

As used herein, the term “computer graphics program” refers to a computer program in memory of a computing device that converts the scanning device data into a 3D image of the surface of the substrate that has been scanned and painted.

As used herein, the term “computer graphic user interface” refers to an electronic device, such as a computer, for displaying or depicting one or more of data, an image, list, or images of the surface of the substrate that has been scanned and painted. Also included is time of exposure, where exposed, distance, efficiency, and the like. In one embodiment, the user interface produces a graphic of the surface of the substrate within different indicators (e.g., colors) to show where it is completed, where it is not completed, where there is not enough exposure, and the like.

The method of the present invention for sterilizing the substrate surface involves selecting a system as described above and sterilizing the surface of the substrate, then looking at the graphic image and data that is simultaneously measured to determine if the entire surface of the substrate surface has been sterilized, as well as other information. In other embodiments, measurement of distance, time, energy of lamps, and the like, are determined in order to tell not only has the entire surface been painted, but also that every area received enough light exposure to result in efficient sterilization, as well as the time to complete the process.

### DRAWINGS

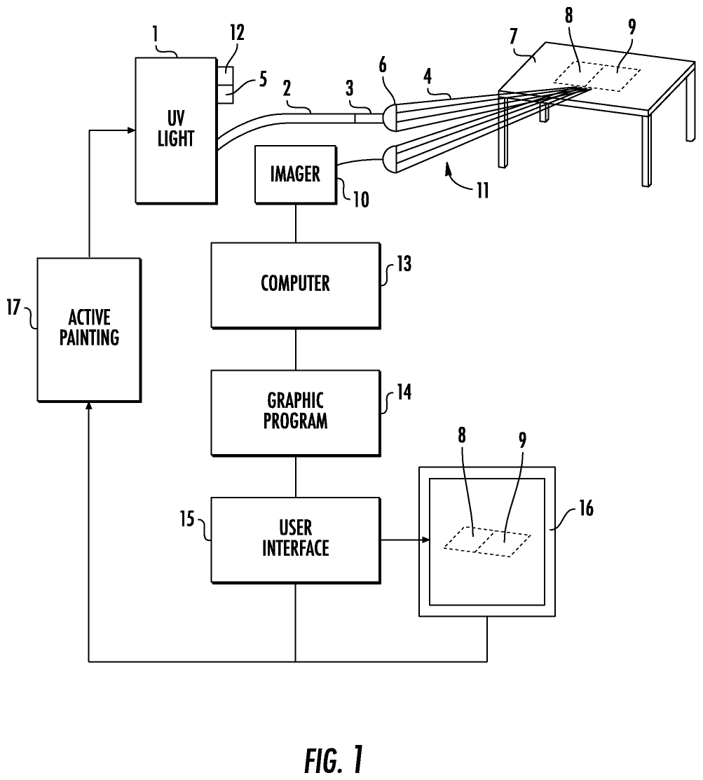

Now referring to the drawings, FIG. 1 is a representation of the system of the invention. In this view, UV light device 1 (low or high energy; with or without heat removed) generates a UV containing light and transmits it via light guide 2 to optical wand 3, wherein UV light 4 is emitted from emitter 6. UV light 4 is shining on table top 7 and creates square pattern of disinfection 8. Next to pattern 8 is previously sterilized area 9.

The imaging device 10 has a digital image, camera, digital scanning device, image, or takes an image with beam 11 in lock register or otherwise positioned with UV light 4 such that both the UV light 4 and the imaging device 10 are shining essentially identically on area 8 at the same time. The system can, in one embodiment, include a timer 12, light energy and distance measuring device 5 to calculate the actual exposure received by the surface painted, time to complete the process, and the like.

The scan from the imaging device 10 is sent to computer 13 and is processed utilizing a standard graphics program 14 which compiles the data received into a graphic representation of the area scanned. This is then transferred to user interface 15, including all data and shown on a monitor 16 or other GUI, to show the areas scanned 8 and 9 and data recording the scan process. The user continues painting with UV light 17 until the graphic shows the entire surface is painted. The program can also include information about how much radiation the surface has received, such that one can determine if every area painted has received sufficient UV light for sterilization as well as time, distance, and light energy.

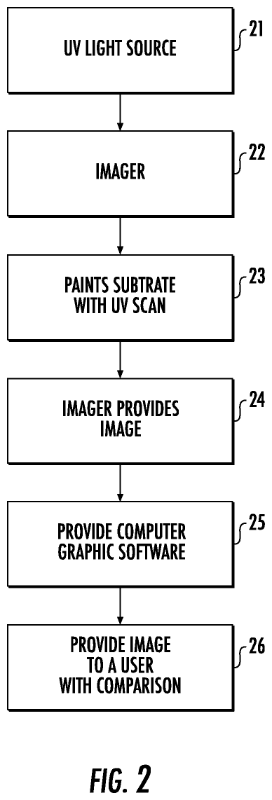

FIG. 2 is a flow chart of the method of the invention. A UV light source 21 with an imager 22 in register with the UV light source 21 from the UV device 1. This UV light paints a substrate 23 while imager measures what has been painted by UV light. The scan obtained is then converted into an image 24. A computer with graphics software 25 is selected and the image is then displayed on the computer for a user 26 showing where the substrate was sterilized and any other data, as described above.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art, still fall within the scope of the invention as claimed by the Applicant.

What is claimed is:

1. A real time system for sterilization of a substrate surface comprising:
a) a UV light source with a UV emitter designed to paint the substrate surface with sterilizing UV light of a given area on the substrate surface when the UV light is shined on the substrate surface;
b) an imaging device having a measuring laser, light, camera, or the like such that the imaging device creates data therefrom;
c) a computer graphics program for converting the scanning device data into an image of the surface of the substrate that has been scanned; and
d) a user interface for depicting the image of the surface of the substrate that has been scanned and creates data about the sterilization.

2. The system according to claim 1 wherein the UV light is polychromatic.

3. The system according to claim 1 wherein the UV light is at least one of UVA, UVB, and UVC.

4. The system according to claim 1 wherein the UV light source is selected from the group consisting of high pressure, medium pressure, low pressure, or LED lamp.

5. The system according to claim 1 wherein the UV emitter is a light guide with a wand type emitter.

6. The system according to claim 1 wherein exposure period, UV light energy, distance to substrate, and time to completion are reported with the image.

7. The system according to claim 1 wherein the user interface is a computer graphic user interface.

8. The system according to claim 1 wherein the imaging device is a 3D imager.

9. A method of sterilizing a substrate surface with UV light comprising:
a) selecting a UV light source with a UV emitter designed to paint the substrate surface with sterilizing UV light of a given area on the substrate surface;
b) selecting an imaging device which when sterilizing the substrate surface with the UV light source is being done, the scanner scans where the substrate has received UV light and creates data therefrom about the method;
c) painting the substrate surface with the UV light source while simultaneously scanning the substrate surface with the imaging device to create data therefrom about the method;
d) providing computer graphics software that converts the imaging device data into an image; and
e) providing the image to a user graphic interface which allows the user to view at least one of what parts of the substrate surface has been scanned, what has not been scanned, and data generated by the imagery device.

10. The method according to claim 9 wherein the UV light is polychromatic.

11. The method according to claim 9 wherein the UV light is at least one of UVA, UVB, and UVC.

12. The method according to claim 9 wherein the UV light source is selected from the group consisting of high pressure, medium pressure, low pressure, or LED lamp.

13. The method according to claim 12 wherein the high energy light source has the heat removed.

14. The method according to claim 9 wherein the UV emitter is a light guide with a wand type emitter.

15. The method according to claim 9 wherein the user interface is a computer graphic user interface.

16. The method according to claim 9 wherein the imaging device is a 3D imager.

17. The system according to claim 1 wherein if the image provided to the user indicates that a portion of the substrate surface has not been painted then the user repeats step c) on the missed parts of the substrate surface.

18. The system according to claim 1 wherein data includes at least one of exposure period, UV light energy, distance to the substrate, and time to completion.
